(19) United States
(12) Patent Application Publication
(10) Pub. No.: US 2024/0277209 A1
(43) Pub. Date: Aug. 22, 2024

(54) URETEROSCOPY IMAGING SYSTEM AND METHODS

(71) Applicants: The Chancellor, Masters and Scholars of the University of Oxford, Oxford (GB); Oxford University Innovation Limited, Oxford (GB); Boston Scientific Scimed Inc., Maple Grove, MN (US)

(72) Inventors: Soumya Gupta, Oxford (GB); Sharib Ali, Chapel Allerton (GB); Jens Rittscher, Kidlington (GB); Benjamin Turney, Oxford (GB); Niraj Prasad Rauniyar, Plymouth, MN (US); Aditi Ray, San Jose, CA (US); Longquan Chen, Lexington, MA (US)

(73) Assignees: The Chancellor, Masters and Scholars of the University of Oxford, Oxford (GB); Oxford University Innovation Limited, Oxford (GB); Boston Scientific Scimed Inc., Maple Grove, MN (US)

(21) Appl. No.: 18/581,220

(22) Filed: Feb. 19, 2024

Related U.S. Application Data
(60) Provisional application No. 63/446,988, filed on Feb. 20, 2023.

(51) Int. Cl. A61B 1/00; A61B 1/307

(52) U.S. Cl. CPC A61B 1/000094; A61B 1/0005; A61B 1/00057; A61B 1/307; A61B 2576/02

(57) ABSTRACT

This disclosure teaches determining a renal calculus size based on calibration data generated from an endoscopic imager. During a lithotripsy procedure, images are received by a deployed endoscopic probe. Based on the received imaging data, the generated calibration data, and known properties of the endoscopic probe, one or more renal calculi in the images are identified and sized. The determined size values are displayed with the images of the renal calculi.

### CROSS-REFERENCE TO RELATED APPLICATIONS

This application claims the benefit of U.S. Provisional Patent Application Ser. No. 63/446,988 filed Feb. 20, 2023, the disclosure of which is incorporated herein by reference.

### TECHNICAL FIELD

The present disclosure generally relates to endoscopy imaging procedures. Particularly, but not exclusively, the present disclosure relates to the processing of renal calculi images during endoscopic lithotripsy.

### BACKGROUND

One procedure to address renal calculi, also known as kidney stones, is ureteral endoscopy, also known as ureteroscopy. A probe with a camera or other sensor is inserted into the patient's urinary tract to find and destroy the calculi. An ideal procedure is one in which the medical professional quickly identifies and smoothly eliminates each of the kidney stones.

Adequately dealing with a kidney stone requires correctly estimating its size, shape, and composition so that the correct tool or tools can be used to identify and address it. Because an endoscopic probe is used rather than the surgeon's own eyes, it may not always be clear from the returned images exactly where or how big each calculus is. A need therefore exists for an imaging system for ureteral endoscopy that provides the operator with automatically-generated information in tandem with the received images.

### BRIEF SUMMARY

This Summary is provided to introduce a selection of concepts in a simplified form that are further described below in the Detailed Description. This Summary is not intended to necessarily identify key features or essential features of the claimed subject matter, nor is it intended as an aid in determining the scope of the claimed subject matter.

The present disclosure provides ureteral endoscopic imaging solutions that address shortcomings in conventional solutions. For example, the systems according to the present disclosure can provide timely information during the lithotripsy procedure by identifying and providing the size of the renal calculi visible on the endoscope image.

In general, the present disclosure provides for calculation and display of renal calculus size during an endoscopic procedure. Endoscopic images of known objects are taken to provide calibration data. Accurate size data is overlaid on display images provided by the deployed endoscopic probe.

In some examples, the present disclosure provides method of endoscopic imaging, comprising: receiving calibration data, the calibration data generated from imaging data taken from an endoscopic probe; receiving, from the endoscopic probe while it is deployed, imaging data that includes at least one image of a renal calculus; applying the calibration data to the imaging data to determine a size of the renal calculus; and displaying the determined size.

In some implementations, displaying the determined size comprises displaying the imaging data with the determined size visually associated with the at least one image of the renal calculus. In some implementations, displaying the determined size comprises displaying the image of the renal calculus labeled with the predetermined size. The method may further comprise displaying a shape overlaid on the image of the renal calculus, wherein the determined size is visually associated with the overlaid shape. In some implementations, the calibration data is generated from at least three images taken of one or more predetermined objects of known dimensions by the endoscopic probe. In some implementations, calibration data comprises a plurality of camera parameters and a plurality of distortion parameters. The method may further comprise determining a focal length for the imaging data based on the calibration data. In some implementations, the imaging data further includes at least one image of a surgical device component having one or more known dimensions, and determining the size of the renal calculus comprises determining a distance from the camera of the deployed endoscopic probe to the renal calculus based on the image and one or more known dimensions of the surgical device component. In some implementations, the surgical device component is a laser fiber. In some implementations, the steps of applying the calibration data to the imaging data to determine a size of the renal calculus and displaying the determined size occur while the endoscopic probe is deployed.

The method may further comprise, while the endoscopic probe is still deployed, receiving additional imaging data including at least one image of the renal calculus; based on the additional imaging data, determining an updated size for the renal calculus; and displaying the updated size. In some implementations, displaying the determined size further comprises comparing the determined size to at least one threshold value; selecting one or more display parameters based on the comparison of the determined size to the at least one threshold value; and displaying the determined size with the selected one or more display parameters. In some implementations, the selected one or more display parameters comprises a color selected from a plurality of colors each associated with a range of size values.

In some embodiments, the present disclosure can be implemented as a computer readable storage medium comprising instructions, which when executed by a processor of a computing device cause the processor to implement any of the methods described herein. With some embodiments, the present disclosure can be implemented as a computer comprising a processor; and memory comprising instructions, which when executed by the processor cause the computing system to implement any of the methods described herein.

In some examples, the present disclosure can be implemented as a computing system, comprising: a processor; a display; and memory comprising instructions, which when executed by the processor cause the computing system to: receive calibration data, the calibration data generated from imaging data taken from an endoscopic probe; receive, from the endoscopic probe while it is deployed, imaging data that includes at least one image of a renal calculus; apply the calibration data to the imaging data to determine a size of the renal calculus; and display the determined size on the display.

In some implementations, the computing system includes instructions, which when executed by the processor, causes the computing system to display the imaging data with the determined size visually associated with the at least one image of the renal calculus. In some implementations, wherein the surgical device component is a laser fiber. In some implementations, the imaging data further includes at least one image of a surgical device component having one or more known dimensions and determining the size of the renal calculus comprises determining a distance from the camera of the deployed endoscopic probe to the renal calculus based on the image and one or more known dimensions of the surgical device component. In some implementations, the computing system includes instructions, which when executed by the processor, causes the computing system to, while the endoscopic probe is still deployed, receive additional imaging data including at least one image of the renal calculus; based on the additional imaging data, determine an updated size for the renal calculus; and display the updated size.

In some examples, the disclosure includes a computer readable storage medium comprising instructions, which when executed by a processor of a computing device causes the processor to receive calibration data, the calibration data generated from imaging data taken from an endoscopic probe; receive, from the endoscopic probe while it is deployed, imaging data that includes at least one image of a renal calculus; apply the calibration data to the imaging data to determine a size of the renal calculus; and display the determined size.

In some embodiments, the computer readable storage medium includes instructions, which when executed by the processor, cause the computing system to display the imaging data with the determined size visually associated with the at least one image of the renal calculus. In some embodiments, the computer readable storage medium includes instructions, which when executed by the processor, cause the computing system to, while the endoscopic probe is still deployed, receive additional imaging data including at least one image of the renal calculus; based on the additional imaging data, determine an updated size for the renal calculus; and display the updated size.

### BRIEF DESCRIPTION OF THE SEVERAL VIEWS OF THE DRAWINGS

To easily identify the discussion of any element or act, the most significant digit or digits in a reference number refer to the figure number in which that element is first introduced.

FIG. 1 illustrates an endoscopic imaging system, in accordance with embodiment(s) of the present disclosure.

FIG. 2 illustrates a logic flow, in accordance with embodiment(s) of the present disclosure.

FIG. 3 illustrates calibration imaging in accordance with embodiments(s) of the present disclosure.

FIG. 4 illustrates a logic flow, in accordance with embodiment(s) of the present disclosure.

FIG. 5A illustrates an endoscopic image, in accordance with embodiment(s) of the present disclosure.

FIG. 5B is a visualization of processing of data taken from the endoscopic image of FIG. 5A, in accordance with embodiment(s) of the present disclosure.

FIG. 5C is a display of an endoscopic image including a renal calculus size in accordance with embodiment(s) of the present disclosure.

### DETAILED DESCRIPTION

The foregoing has broadly outlined the features and technical advantages of the present disclosure such that the following detailed description of the disclosure may be better understood. It is to be appreciated by those skilled in the art that the embodiments disclosed may be readily utilized as a basis for modifying or designing other structures for carrying out the same purposes of the present disclosure. The novel features of the disclosure, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

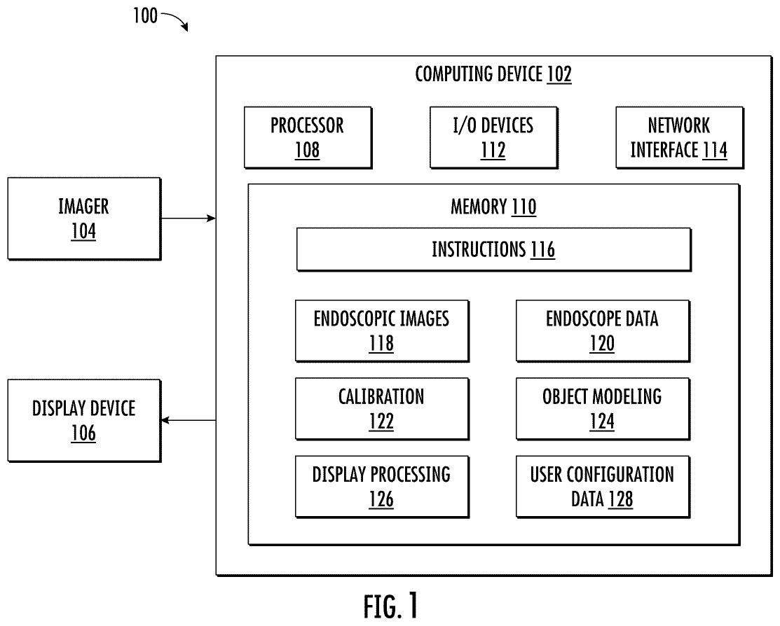

FIG. 1 illustrates an endoscopic imaging system 100, in accordance with non-limiting examples of the present disclosure. In general, an endoscopic imaging system 100 is a system for processing and displaying images in real-time that are captured by an endoscope deployed for lithotripsy. Although the present disclosure focuses of lithotripsy, which would use a ureteroscope, the disclosure is applicable to other endoscopic modalities, such as, for example, colonoscopes, bronchoscopes, etc.

Endoscopic imaging system 100 includes a computing device 102. Optionally, endoscopic imaging system 100 includes imager 104 and display device 106. In an example, computing device 102 can receive an image or a group of images representing a patient's urinary tract. For example, computing device 102 can receive endoscopic images 118 from imager 104. In some embodiments, imager 104 can be a camera or other sensor deployed with an endoscope during a lithotripsy procedure.

Although the disclosure uses visual-spectrum camera images to describe illustrative embodiments, imager 104 can be any endoscopic imaging device, such as, for example, a fluoroscopy imaging device, an ultrasound imaging device, an infrared or ultraviolet imaging device, a computed tomography (CT) imaging device, a magnetic resonance (MR) imaging device, a positron emission tomography (PET) imaging device, or a single-photon emission computed tomography (SPECT) imaging device.

Imager 104 can generate information elements, or data, including indications of renal calculi. Computing device 102 is communicatively coupled to imager 104 and can receive the data including the endoscopic images 118 from imager 104. In general, endoscopic images 118 can include indications of shape data and/or appearance data of the urinary tract. Shape data can include landmarks, surfaces, and boundaries of the three-dimensional surfaces of the urinary tract. With some examples, endoscopic images 118 can be constructed from two-dimensional (2D) or three-dimensional (3D) images.

In general, display device 106 can be a digital display arranged to receive rendered image data and display the data in a graphical user interface. Computing device 102 can be any of a variety of computing devices. In some embodiments, computing device 102 can be incorporated into and/or implemented by a console of display device 106. With some embodiments, computing device 102 can be a workstation or server communicatively coupled to imager 104 and/or display device 106. With still other embodiments, computing device 102 can be provided by a cloud-based computing device, such as, by a computing as a service system accessibly over a network (e.g., the Internet, an intranet, a wide area network, or the like). Computing device 102 can include processor 108, memory 110, input and/or output (I/O) devices 112, and network interface 114.

The processor 108 may include circuitry or processor logic, such as, for example, any of a variety of commercial processors. In some examples, processor 108 may include multiple processors, a multi-threaded processor, a multi-core processor (whether the multiple cores coexist on the same or separate dies), and/or a multi-processor architecture of some other variety by which multiple physically separate processors are in some way linked. Additionally, in some examples, the processor 108 may include graphics processing portions and may include dedicated memory, multiple-threaded processing and/or some other parallel processing capability. In some examples, the processor 108 may be an application specific integrated circuit (ASIC) or a field programmable integrated circuit (FPGA).

The memory 110 may include logic, a portion of which includes arrays of integrated circuits, forming non-volatile memory to persistently store data or a combination of non-volatile memory and volatile memory. It is to be appreciated, that the memory 110 may be based on any of a variety of technologies. In particular, the arrays of integrated circuits included in memory 110 may be arranged to form one or more types of memory, such as, for example, dynamic random access memory (DRAM), NAND memory, NOR memory, or the like.

I/O devices 112 can be any of a variety of devices to receive input and/or provide output. For example, I/O devices 112 can include, a keyboard, a mouse, a joystick, a foot pedal, a display (e.g., touch, non-touch, or the like) different from display device 106, a haptic feedback device, an LED, or the like. One or more features of the endoscope may also provide input to the imaging system 100.

Network interface 114 can include logic and/or features to support a communication interface. For example, network interface 114 may include one or more interfaces that operate according to various communication protocols or standards to communicate over direct or network communication links. Direct communications may occur via use of communication protocols or standards described in one or more industry standards (including progenies and variants). For example, network interface 114 may facilitate communication over a bus, such as, for example, peripheral component interconnect express (PCIe), non-volatile memory express (NVMe), universal serial bus (USB), system management bus (SMBus), SAS (e.g., serial attached small computer system interface (SCSI)) interfaces, serial AT attachment (SATA) interfaces, or the like. Additionally, network interface 114 can include logic and/or features to enable communication over a variety of wired or wireless network standards (e.g., 802.11 communication standards). For example, network interface 114 may be arranged to support wired communication protocols or standards, such as, Ethernet, or the like. As another example, network interface 114 may be arranged to support wireless communication protocols or standards, such as, for example, Wi-Fi, Bluetooth, ZigBee, LTE, 5G, or the like.

Memory 110 can include instructions 116 and endoscopic images 118. During operation, processor 108 can execute instructions 116 to cause computing device 102 to receive endoscopic images 118 from imager 104 via input and/or output (I/O) devices 112. Processor 108 can further execute instructions 116 to identify urinary structures and material in need of removal. Further still, processor 108 can execute instructions 116 to generate images to be displayed on display device 106.

Memory 110 can further include endoscope data 120 that provides information about endoscope systems, including visible endoscope components; a calibration module 122 that guides an automated system and/or a system user to capture the images necessary to generate calibration data; an object modeling module 124 capable of determining an object's size from the received endoscopic images 118; a display processing module 126 including the tools necessary to augment endoscopic images with the calculated values; and user configuration data 128 to reference when customizable options are made available to a user.

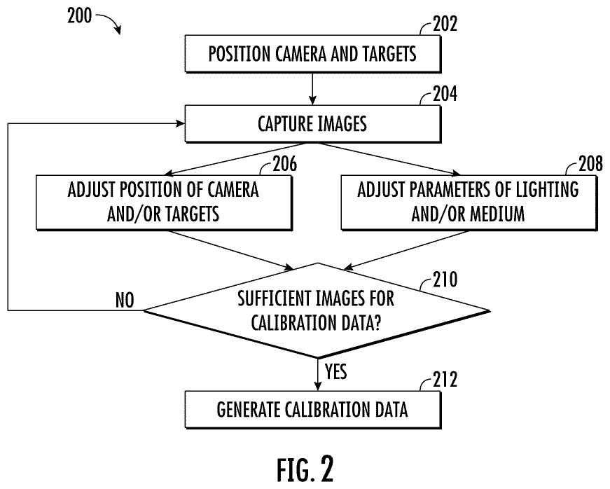

The above is described in greater detail below, such as, for example, in conjunction with logic flow 200 from FIG. 2. With some examples, endoscopic imaging system 100 can be provided with just computing device 102. That is, endoscopic imaging system 100 can include computing device 102 and a user of endoscopic imaging system 100 can provide imager 104 and display device 106, which are compatible with computing device 102.

It is noted that endoscopic imaging system 100 includes custom components, which are specifically configured, programmed, and/or arranged to carry out the logic flows and methods detailed herein. For example, processor 108 can be preconfigured to execute object recognition and graphical processing operations as further described.

FIG. 2 illustrates logic flow 200, according to some embodiments of the present disclosure. Logic flow 200 can be implemented to generate calibration data for an endoscopic imaging and process based on automated detection of renal calculi. Although logic flow 200 is described with reference to endoscopic imaging system 100 and FIG. 1, examples are not limited by this and logic flow 200 could be implemented by a system having components not depicted in FIG. 1.

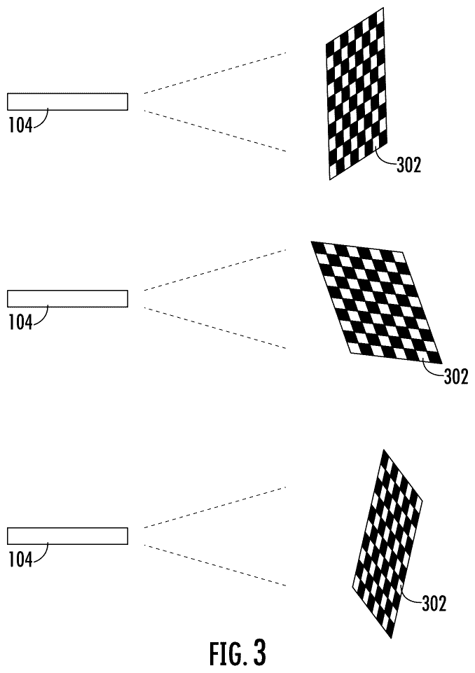

Logic flow 200 can begin at block 202, “position camera and targets.” An endoscopic camera or other sensor used in an endoscopic lithotripsy is positioned in front of one or more objects with known visual characteristics, such as the checkerboard pattern described and discussed below with respect to FIG. 3.

At block 204, the system captures images of the positioned objects. In block 206, after each set of images, the position of the camera and/or of one or more of the objects is adjusted as needed for image processing.

At block 208, the system may also adjust the parameters of the visual environment of the images. For example, lighting conditions may be varied. The camera, the target objects, or both may be immersed in a medium such as water to better account for the refractive index in the environment into which the endoscope will be deployed.

At decision block 210, the system evaluates whether the captured images are sufficiently clear and varied to generate calibration data. If the images are not sufficient, further images may be taken as described above. If the images are suitable to use for calibration, then calibration data is generated for use during subsequent operations.

One of ordinary skill will recognize that the frequency and specifics of calibration may vary according to the needs of the operator and the capabilities of device. It is contemplated that, in some implementations, the logic described above may represent “factory calibration” that is performed when an endoscope or endoscope camera is first assembled and tested. The calibration data may be stored in memory local to the endoscope device or in another accessible memory location, such as part of the endoscope imaging system.

For some devices and/or some applications, it may be necessary to provide calibration data frequently. For some applications, it may be important to provide calibration data within a short duration before the use of the imager. The system can provide guidance to the user of certain calibration data is necessary for certain functions of the display.

The resulting calibration data may vary in precision and format according to the nature of the device and the needs of the system. Calibration data may include, as one example, values representing distortion along one or more spatial axes. Intensity modulation, transparency, skew, and color correction may all be included in the calibration data, conforming the camera image more closely to the imaged object. The calibration may also determine relevant information for the imager, such as field of view and focal length.

In one specific example, the camera calibration data may include a matrix A of parameters:

$$A = [ \begin{matrix}α & γ & {u}_{0} \\ 0 & β & {v}_{0} \\ 0 & 0 & 1\end{matrix} ]$$

Where α and β are scale factors for the two images axes, γ is the skewness parameter for the two image axes, and (u₀, v₀) is the coordinate of the principal point used in the calibration. In addition to these values, the system may generate k1 and k2 values, representing coefficients of radial distortion.

FIG. 3 shows an imager 104, such as an endoscope camera, placed in front of an object target 302 for calibration. The object target 302 is a planar image with known size, shape, and geometry. Each of the figures shows the same object target 302 placed in a different orientation relative to the imager 104. The distance, position, and angle of the object target 302 are known. Comparing the shape and size of the known object target 302 to the images produced by the imager 104 allows the system to determine calibration parameters for the imager 104.

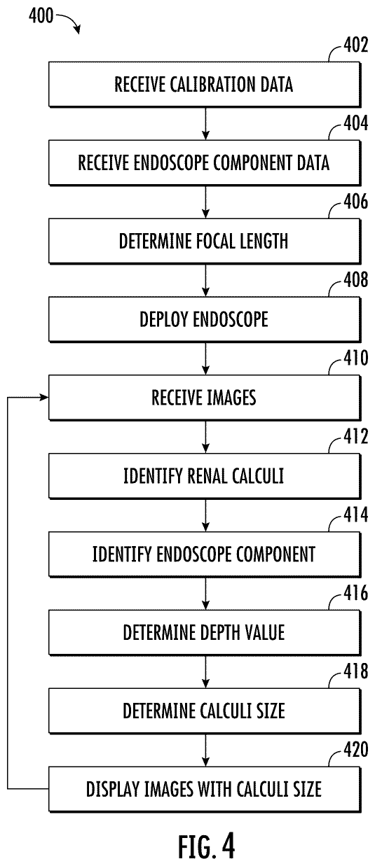

FIG. 4 illustrates logic flow 400, according to some embodiments of the present disclosure. Logic flow 400 can be implemented to process and display imaging data during an endoscopic procedure. Although logic flow 400 is described with reference to endoscopic imaging system 100 and FIG. 1, examples are not limited by this and logic flow 400 could be implemented by a system having components not depicted in FIG. 1.

At block 402, the system 100 receives calibration data, which may have been stored in system memory 110 when the calibration data was generated if it was generated locally or may be accessed from another system.

At block 404, the system 100 receives data regarding one or more endoscopic components that will be visible on camera for use in image processing. In some implementations, the visible component may be a laser fiber. The data may be provided based on the endoscope model and configuration. In some embodiments, the size and shape of the laser fiber may be generated based on the calibration images described with respect to logic flow 200 and FIGS. 2 and 3. In some embodiments, a user may be prompted to manually input data regarding the laser fiber or another endoscopic component.

As noted above with respect to calibration, focal length may be one value included in the received calibration data. Alternatively, at block 406, the system 100 calculates the focal length from the received data and/or information about the endoscope imager.

At block 408, the endoscope is deployed as part of a lithotripsy procedure. Techniques of lithotripsy vary based on the needs of the patient and the available technology. Any part of the urinary tract may be the destination of the endoscope, and the procedure may target renal calculi in different locations during a single procedure.

The remainder of the logic flow 400, representing blocks 410-420, takes place while the endoscope is deployed and the lithotripsy procedure is carried out. These steps 410-420 represent an image being captured, processed, and displayed to the medical professional performing the procedure. The displayed images are “live,” meaning the delay between receiving and displaying the images is short enough that the images can be used by the professional to control the lithotripsy tools in real time based on what is displayed.

At block 410, the system receives one or more endoscopic images from the imager. The system may process more than one image at once, based on expected processing latency as well as the imager's rate of capturing images; each individual image is referred to as a “frame” and the speed of capture as the “framerate”. For example, where the system might take as much as 0.1 seconds to process a set of frames and the imager has a framerate of 50 frames per second, the system might process 5 or more frames as a set to have sufficient throughput to process the available data. The system may also process fewer than all the received frames; in some implementations it may select a fraction of the received frames such as every second or third frame to process.

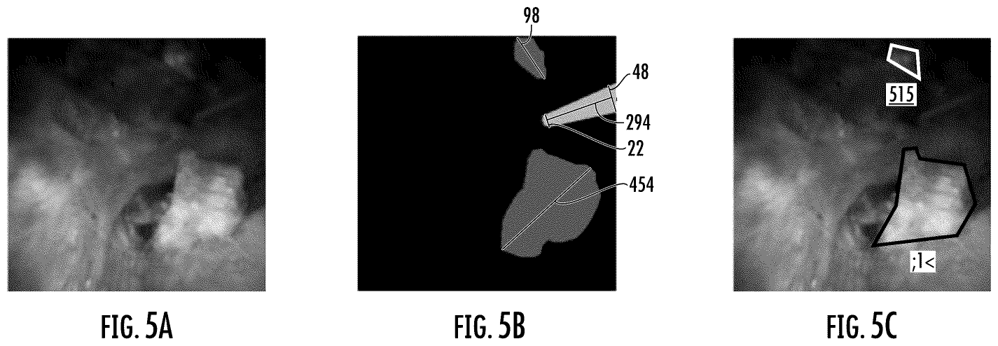

FIG. 5A illustrates an endoscopic image 500, representing a single “frame” captured by an imager during deployment of an endoscope for lithotripsy. Although shown in greyscale for FIG. 5A, the system receives and processes a color image (which may, for example, represent a standard RGB or CMYK encoding, or any other image format that can be read and displayed). Two renal calculi and a laser fiber are visible in the image.

At block 412, the system identifies renal calculi in the received endoscopic images. The calculus may be identified by shape, size, coloration, movement relative to the background, or any combination of these. The movement of the endoscope by the medical practitioner may also affect identification of a renal calculus. For example, an object that is centered in the imager's field of view for multiple seconds may be identified as a renal calculus, while a similar object that is quickly moved out of the field of view may not be. In some implementations, the system may limit the identification only to those calculi that take up a certain portion of the field of view to avoid the expenditure of resources on fragments below a threshold size or those that are too far from the endoscopic probe.

Various image-recognition processes may be used to ensure object persistence, so that once an image is positively identified as a renal calculus, subsequently received frames identify the same portion of the image as a renal calculus. When an image portion of the same color, position, and/or shape as an identified calculus is found on a later frame, it is preferentially identified as a renal calculus. In other implementations, where no such persistence is used, the system may require the same initial recognition processes be used in each subsequent set of frames, which may more quickly end the mismarking of false positives.

FIG. 5B is a representation of the visual processing performed by the system 100 to isolate the measured system elements in the image. Image portions 502a and 502b are each identified as renal calculi. The system identifies the size of the image portions as 454 pixels and 98 pixels respectively.

In the presented example, the size measurement represents the maximum length between any two points in the identified portion of the image. Other methods of generating the size are known, such as taking the portion height (the difference between the maximum and minimum y coordinates of pixels in the portion) or width (the difference in the x coordinates).

It is customary for a single length value to be used when considering the size of renal calculi, but the system is not limited to this value. Size values could instead be assessed in two or three dimensions, representing a cross-sectional area or volume of the object, respectively. For example, a total number of pixels of the identified image portion could be used as an estimate of the cross-sectional area of the calculus. A model, taking the known size values as a diameter or cross section of a sphere, could further be used to extrapolate a three-dimensional volume in pixels for the portion of the image.

At block 414, the system identifies one or more endoscopic components within the received images. As with identifying renal calculi, shape, size, coloration, and relative movement can all be used to identify the component, which may be a laser fiber, a grasper, or any other tool used in the lithotripsy operation. In many implementations, a component will be used that is expected to stay in the imager's field of view throughout the procedure. The system may again presume image persistence to preferentially identify the component as remaining in the same area of the image in subsequent frames.

In FIG. 5B, image portion 504 is identified as a laser fiber. The portion of the laser fiber visible in the image is 294 pixels long, tapering from a width of 48 pixels at the edge of the image to 22 pixels at the tip.

At block 416, the system determines a depth value to use for the renal calculi based on the known size and image dimensions of the endoscopic component. In the example of the image portion 504 identified as a laser fiber, the proportion of the width measurements provides an angle for the fiber relative to the imager, and the length in pixels can be used to determine approximately how deep the fiber stretches. This depth can then be applied as an approximate depth for the renal calculi in the image.

At block 418, the size of each identified renal calculus is determined based on the received and calculated image data. The size may, in some implementations, be calculated by the following formula:

$$s = ( p / r ) * ( d / f )$$

Where s is the size of the calculus (in mm), p is the size of the calculus image (in pixels), r is the resolution of the image (in pixels per mm), d is the depth value used for the calculus (in mm), and f is the focal length of the imager (in mm).

At block 420, the calculated size value for each renal calculus is added to the image display. This may be done by overlaying the number on a portion of the renal calculus image itself or by placing it nearby. The portion of the image may be highlighted, and a tooltip or other marking may be included. In some implementations, the color of the size value on the display may vary according to the calculated size: for example, green for calculi below 3 mm, yellow for calculi between 3 and 6 mm, and red for calculi above 6 mm in size. These threshold values, as well as other aspects of the size display, may be customizable in settings available to users of the system.

FIG. 5C illustrates an example of a display image 510 with added markings 512a and 512b showing the size of the renal calculi. A rough polygonal outline of each image is displayed along with a label denoting the size of the object in millimeters. For the larger renal calculus, the markings 512a are in black, while the markings 512b for the smaller renal calculus are in white. The color of the markings may represent some aspect of the object, such as its size relative to a threshold, but may also be selected either automatically or manually based on other factors of the display such as contrast for legibility.

The system may include a timer before the displayed values are changed. For example, even if the system processes a new set of frames 10 times per second, once a value has been determined and output for display, it may be 1 second before that display value can be changed. This is to avoid the display fluctuating so rapidly as to reduce its value to the users.

Terms used herein should be accorded their ordinary meaning in the relevant arts, or the meaning indicated by their use in context, but if an express definition is provided, that meaning controls.

Herein, references to “one embodiment” or “an embodiment” do not necessarily refer to the same embodiment, although they may. Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively, unless expressly limited to one or multiple ones. Additionally, the words “herein,” “above,” “below” and words of similar import, when used in this application, refer to this application as a whole and not to any portions of this application. When the claims use the word “or” in reference to a list of two or more items, that word covers all the following interpretations of the word: any of the items in the list, all the items in the list and any combination of the items in the list, unless expressly limited to one or the other. Any terms not expressly defined herein have their conventional meaning as commonly understood by those having skill in the relevant art(s).

What is claimed is:

1. A method of endoscopic imaging, comprising:
receiving calibration data, the calibration data generated from imaging data taken from an endoscopic probe;
receiving, from the endoscopic probe while it is deployed, imaging data that includes at least one image of a renal calculus;
applying the calibration data to the imaging data to determine a size of the renal calculus; and
displaying the determined size.

2. The method of claim 1, wherein displaying the determined size comprises displaying the imaging data with the determined size visually associated with the at least one image of the renal calculus.

3. The method of claim 1, wherein displaying the determined size comprises displaying the image of the renal calculus labeled with the predetermined size.

4. The method of claim 3, further comprising:
displaying a shape overlaid on the image of the renal calculus, wherein the determined size is visually associated with the overlaid shape.

5. The method of claim 1, wherein the calibration data is generated from at least three images taken of one or more predetermined objects of known dimensions by the endoscopic probe.

6. The method of claim 1, wherein the calibration data comprises a plurality of camera parameters and a plurality of distortion parameters.

7. The method of claim 1, further comprising:
determining a focal length for the imaging data based on the calibration data.

8. The method of claim 1,
wherein the imaging data further includes at least one image of a surgical device component having one or more known dimensions, and
wherein determining the size of the renal calculus comprises determining a distance from the camera of the deployed endoscopic probe to the renal calculus based on the image and one or more known dimensions of the surgical device component.

9. The method of claim 8, wherein the surgical device component is a laser fiber.

10. The method of claim 1, wherein the steps of applying the calibration data to the imaging data to determine a size of the renal calculus and displaying the determined size occur while the endoscopic probe is deployed.

11. The method of claim 1, further comprising:
while the endoscopic probe is still deployed, receiving additional imaging data including at least one image of the renal calculus;
based on the additional imaging data, determining an updated size for the renal calculus; and
displaying the updated size.

12. The method of claim 1, wherein displaying the determined size further comprises:
comparing the determined size to at least one threshold value;
selecting one or more display parameters based on the comparison of the determined size to the at least one threshold value; and
displaying the determined size with the selected one or more display parameters.

13. The method of claim 12, wherein the selected one or more display parameters comprises a color selected from a plurality of colors each associated with a range of size values.

14. A computing system, comprising:
a processor;
a display; and
memory comprising instructions, which when executed by the processor cause the computing system to:
receive calibration data, the calibration data generated from imaging data taken from an endoscopic probe;
receive, from the endoscopic probe while it is deployed, imaging data that includes at least one image of a renal calculus;
apply the calibration data to the imaging data to determine a size of the renal calculus; and
display the determined size on the display.

15. The computing system of claim 14, the instruction, when executed by the processor, causes the computing system to display the imaging data with the determined size visually associated with the at least one image of the renal calculus.

16. The method of claim 15, wherein the surgical device component is a laser fiber.

17. The computing system of claim 14,
wherein the imaging data further includes at least one image of a surgical device component having one or more known dimensions, and
wherein determining the size of the renal calculus comprises determining a distance from the camera of the deployed endoscopic probe to the renal calculus based on the image and one or more known dimensions of the surgical device component.

18. A computer readable storage medium comprising instructions, which when executed by a processor of a computing device cause the processor to:
receive calibration data, the calibration data generated from imaging data taken from an endoscopic probe;
receive, from the endoscopic probe while it is deployed, imaging data that includes at least one image of a renal calculus;
apply the calibration data to the imaging data to determine a size of the renal calculus; and
display the determined size.

19. The computer readable storage medium of claim 18, the instruction, when executed by the processor, causes the computing system to display the imaging data with the determined size visually associated with the at least one image of the renal calculus.

20. The computer readable storage medium of claim 18, the instruction, when executed by the processor, causes the computing system to:
while the endoscopic probe is still deployed, receive additional imaging data including at least one image of the renal calculus;
based on the additional imaging data, determine an updated size for the renal calculus; and
display the updated size.
